(19) United States
(12) Patent Application Publication
(10) Pub. No.: US 2011/0224214 A1
(43) Pub. Date: Sep. 15, 2011

(54) PROCESS FOR PREPARING POLYMORPH

(75) Inventor: William L. ROCCO, Reading, PA (US)

(73) Assignee: SANOFI-AVENTIS U.S. LLC, Bridgewater, NJ (US)

(21) Appl. No.: 13/027,699

(22) Filed: Feb. 15, 2011

Related U.S. Application Data
(63) Continuation of application No. PCT/US2009/053698, filed on Aug. 13, 2009.
(60) Provisional application No. 61/089,648, filed on Aug. 18, 2008.

(51) Int. Cl. A61K 31/5025; C07D 487/04

(52) U.S. Cl. 514/248; 544/234

(57) ABSTRACT

Disclosed is a novel process for preparing crystalline Form II of 7-chloro-N,N,5-trimethyl-4-oxo-3-phenyl-3,5-dihydro-4H-pyridazino[4,5-b]indole-1-acetamide.

### CROSS-REFERENCE TO RELATED APPLICATIONS

This application is a continuation of International Application No. PCT/US2009/053698, filed Aug. 13, 2009, which claims the benefit of priority of U.S. Provisional Application No. 61/089,648, filed Aug. 18, 2008, both of which are incorporated herein by reference.

### FIELD OF THE INVENTION

The present invention is directed to a novel process for preparing crystalline Form II of 7-chloro-N,N,5-trimethyl-4-oxo-3-phenyl-3,5-dihydro-4H-pyridazino[4,5-b]indole-1-acetamide.

### BACKGROUND OF THE INVENTION

7-Chloro-N,N,5-trimethyl-4-oxo-3-phenyl-3,5-dihydro-4H-pyridazino[4,5-b]indole-1-acetamide, which has the structure of Formula (A):

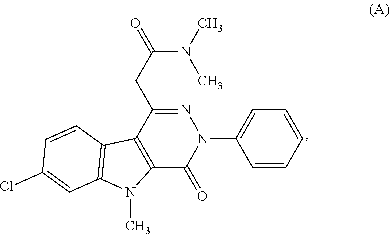

possesses a high affinity for the peripheral-type benzodiazepine receptors, and is known to be useful for the treatment of neurodegenerative diseases. The preparation, physical properties and beneficial pharmacological properties of 7-chloro-N,N,5-trimethyl-4-oxo-3-phenyl-3,5-dihydro-4H-pyridazino[4,5-b]indole-1-acetamide are described in, for example, U.S. Pat. No. 6,262,045 (also WO99/06406) and, in particular, U.S. Pat. No. 6,395,729 (also WO00/44384), both of which are incorporated by reference in their entirety. The processes described in these patents result in the isolation of 7-chloro-N,N,5-trimethyl-4-oxo-3-phenyl-3,5-dihydro-4H-pyridazino[4,5-b]indole-1-acetamide in one defined crystalline form, herein designated as Form I, which has limited solubility.

WO2007/027525 describes a novel crystalline form of 7-chloro-N,N,5-trimethyl-4-oxo-3-phenyl-3,5-dihydro-4H-pyridazino[4,5-b]indole-1-acetamide, designated as crystalline Form II, and processes for preparing crystalline Form II.

In order to render possible the development of formulations comprising crystalline Form II of 7-chloro-N,N,5-trimethyl-4-oxo-3-phenyl-3,5-dihydro-4H-pyridazino[4,5-b]indole-1-acetamide, it is necessary to find a method for preparing crystalline Form II which is reproducible and which gives rise to Form II of the required degree of polymorphic purity in a rapid and efficient manner.

### SUMMARY OF THE INVENTION

The present invention provides a novel process for preparing crystalline Form II of 7-chloro-N,N,5-trimethyl-4-oxo-3-phenyl-3,5-dihydro-4H-pyridazino[4,5-b]indole-1-acetamide, which minimizes the conversion of crystalline Form II to crystalline Form I by utilizing spray drying techniques.

### BRIEF DESCRIPTION OF THE DRAWINGS

FIG. 1 is an overlay of X-ray powder diffractograms of crystalline Form I and crystalline Form II of 7-chloro-N,N,5-trimethyl-4-oxo-3-phenyl-3,5-dihydro-4H-pyridazino[4,5-b]indole-1-acetamide, including crystalline Form II prepared according to aspects of the present invention.

FIG. 2 is a Fourier Transform Infrared (FTIR) spectrum comparing crystalline Form I and crystalline Form II of 7-chloro-N,N,5-trimethyl-4-oxo-3-phenyl-3,5-dihydro-4H-pyridazino[4,5-b]indole-1-acetamide, including crystalline Form II prepared according to aspects of the present invention.

FIG. 3 is an overlay of Differential Scanning calorimetry thermograms of crystalline Form II of 7-chloro-N,N,5-trimethyl-4-oxo-3-phenyl-3,5-dihydro-4H-pyridazino[4,5-b]indole-1-acetamide, including crystalline Form II prepared according to aspects of the present invention.

FIG. 4 is an overlay of X-ray powder diffractograms comparing crystalline Form II of 7-chloro-N,N,5-trimethyl-4-oxo-3-phenyl-3,5-dihydro-4H-pyridazino[4,5-b]indole-1-acetamide as previously described and crystalline Form II prepared according to aspects of the present invention.

FIG. 5 is an overlay of X-ray powder diffractograms comparing crystalline Form I and crystalline Form II of 7-chloro-N,N,5-trimethyl-4-oxo-3-phenyl-3,5-dihydro-4H-pyridazino[4,5-b]indole-1-acetamide as previously described, and crystalline Form II prepared according to an aspect of the present invention.

FIG. 6 is an overlay of X-ray powder diffractograms comparing crystalline Form I and crystalline Form II of 7-chloro-N,N,5-trimethyl-4-oxo-3-phenyl-3,5-dihydro-4H-pyridazino[4,5-b]indole-1-acetamide as previously described, and a pharmaceutical formulation containing crystalline Form II prepared according to an aspect of the present invention.

### DETAILED DESCRIPTION OF THE INVENTION

### Definitions and Abbreviations

As used above, and throughout the description of the invention, the following abbreviations, unless otherwise indicated, shall be understood to have the following meanings:
DCM dichloromethane
DSC differential scanning calorimetry
EtOH ethanol
g gram
HPLC high performance liquid chromatography
mg milligram
mL milliliter
mm millimeter
XRPD x-ray power diffractometry

As used above, and throughout the description of the invention, the following terms, unless otherwise indicated shall be understood to have the following meanings.

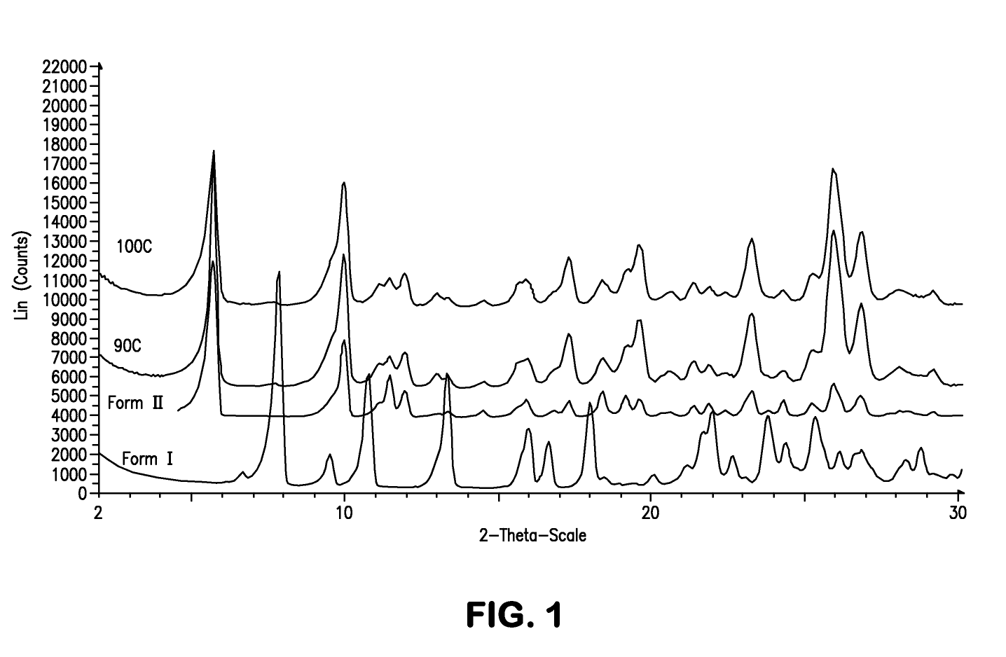

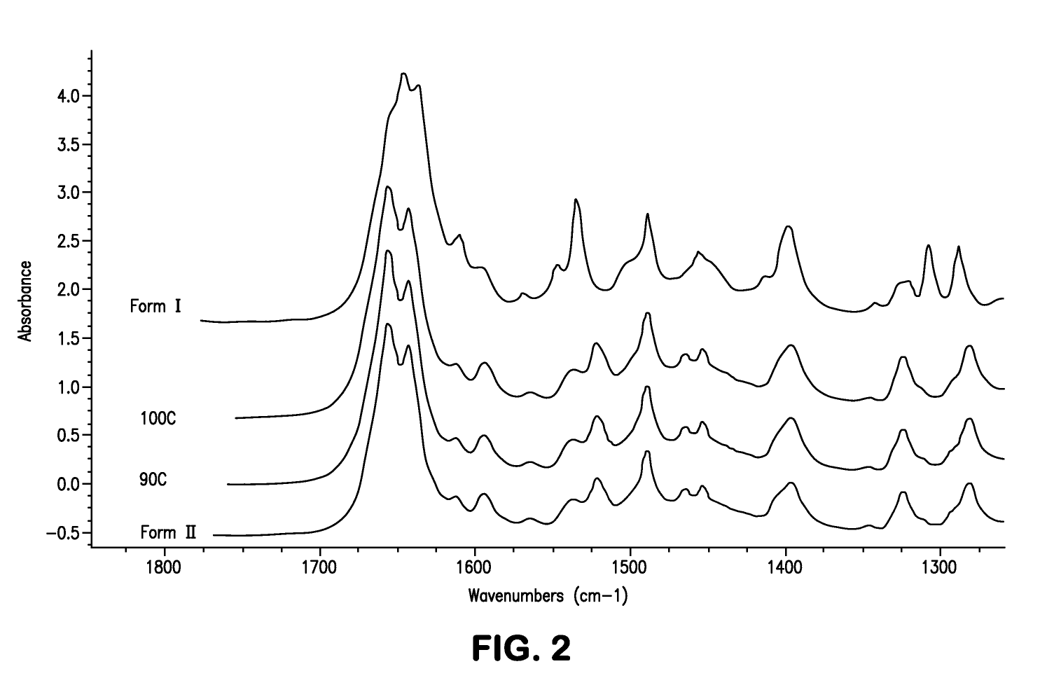

“Form I,” as used herein, is meant to describe a crystalline form of 7-chloro-N,N,5-trimethyl-4-oxo-3-phenyl-3,5-dihydro-4H-pyridazino[4,5-b]indole-1-acetamide that may be characterized using distinguishing data as described herein. Exemplary data is found in FIGS. 1 and 2.

“Form II,” as used herein, is meant to describe a crystalline form of 7-chloro-N,N,5-trimethyl-4-oxo-3-phenyl-3,5-dihydro-4H-pyridazino[4,5-b]indole-1-acetamide that may be characterized using distinguishing data as described herein. Exemplary data is found in FIGS. 1 and 2.

The term “drug substance,” as used herein, refers to 7-chloro-N,N,5-trimethyl-4-oxo-3-phenyl-3,5-dihydro-4H-pyridazino[4,5-b]indole-1-acetamide.

The term “ambient temperature” or “room temperature” refers to a temperature in the range of about 20° C. to about 30° C.

The ability of a substance to exist in more than one crystal form is defined as polymorphism; the different crystal forms of a particular substance are referred to as “polymorphs.” In general, polymorphism is affected by the ability of a molecule of a substance to change its conformation or to form different intermolecular or intra-molecular interactions, particularly hydrogen bonds, which is reflected in different atomic arrangements in the crystal lattices of different polymorphs. In contrast, the overall external form of a substance is known as “morphology,” which refers to the external shape of the crystal and the planes present, without reference to the internal structure. Crystals can display different morphology based on different conditions, such as, for example, growth rate, stirring, and the presence of impurities.

The different polymorphs of a substance may possess different energies of the crystal lattice and, thus, in solid state they may show different physical properties such as density, melting point, color, stability, solubility, et cetera which may, in turn, affect the stability, dissolution rate and/or bioavailability of a given polymorph and its suitability for use as a pharmaceutical and in pharmaceutical compositions.

Crystalline Form II of 7-chloro-N,N,5-trimethyl-4-oxo-3-phenyl-3,5-dihydro-4H-pyridazino[4,5-b]indole-1-acetamide has advantages over Form I. For example, Form II has greater solubility and higher dissolution rates than those of crystalline Form I. It is generally known that polymorphic forms having greater solubility and dissolution rates are usually less physically stable than those having lesser solubility and dissolution rates. Nevertheless, crystalline Form II has a sufficient stability profile to support its use in a pharmaceutical product.

The present invention provides a novel process for preparing crystalline Form II of 7-chloro-N,N,5-trimethyl-4-oxo-3-phenyl-3,5-dihydro-4H-pyridazino[4,5-b]indole-1-acetamide, which provides for rapid solvent removal and minimizes the risk of conversion of the solid from Form II to Form I.

According to the present invention, a process for preparing crystalline Form II of 7-chloro-N,N,5-trimethyl-4-oxo-3-phenyl-3,5-dihydro-4H-pyridazino[4,5-b]indole-1-acetamide comprises spray drying a solution of the drug substance to prepare crystalline Form II.

As used herein, “spray drying” means to atomize a solution or dispersion of 7-chloro-N,N,5-trimethyl-4-oxo-3-phenyl-3,5-dihydro-4H-pyridazino[4,5-b]indole-1-acetamide to form a fine mist of droplets, which immediately enter a drying chamber where they contact a drying gas. The solvent evaporates from the droplets into the drying gas to solidify the droplets. The solid is separated from the drying gas and isolated.

A “drying chamber” as used herein, refers to a chamber (e.g., a vessel, tank, tubing, or coil) which utilizes uses hot gases, such as forced air, nitrogen, nitrogen-enriched air, or argon, to dry particles. The solid may then be isolated in a particle separator, such as a cyclone.

The term “cyclone” as used herein refers to a vortex separator that uses rotational effects and gravity to separate mixtures of solids and/or fluids.

In one aspect, the spray drying process of the present invention comprises dispersing or dissolving the drug substance in a suitable solvent to form a feed solution and pumping the feed solution through a nozzle (atomizer) into a solvent removal system wherein the solvent is removed to form the solid powder.

In another aspect of the invention, at least a potion of the solvent is removed in a drying chamber and the solid is further dried and/or annealed in a fluid bed chamber, tumble dryer, or by any other method generally used in the art which utilizes direct or indirect heating within a static, moving, or fluidized solid bed.

Another aspect of the invention comprises preparing the feed solution of the drug substance at a temperature between about ambient temperature and a temperature below the boiling point of the solvent, particularly around ambient temperature.

The temperature of the inlet gas within the spray dryer can be controlled. Preferably, the inlet temperature of the spray dryer is between about 50° C. and about 130° C., more preferably between about 90° C. and about 120° C. At lower temperatures, such as around 50° C., the solid which is produced may contain a mixture of Form II and non-crystalline material at the time of isolation.

The non-crystalline material can be converted to crystalline Form II upon aging or annealing. Accordingly, one aspect of the invention includes the step of aging or annealing the spray dried material.

The solid is aged or annealed by being held at a temperature in the range from approximately ambient temperature to about 100° C., for example in the range of about 20° C. to about 90° C. The solid is maintained under such conditions until the solid is substantially free of any other polymorph, including non-crystalline forms of 7-chloro-N,N,5-trimethyl-4-oxo-3-phenyl-3,5-dihydro-4H-pyridazino[4,5-b]indole-1-acetamide other than Form II.

Annealing may be accomplished, for example, in a typical oven or within a fluid bed.

The feed solution can be atomized by conventional means well known in the art, such as a two-fluid sonicating nozzle, a two-fluid non-sonicating nozzle, a rotary atomizer, and the like.

A “suitable solvent,” as used herein, is a solvent or mixture of solvents in which the drug substance has adequate solubility, e.g. solubility that is greater than about 1 mg/mL. Examples of suitable solvents include dichloromethane, chloroform, ethanol, methanol, 2-propanol, ethyl acetate, acetone, dimethyl acetamide, or mixtures thereof. A preferred suitable solvent comprises a mixture of dichloromethane and ethanol. A particular suitable solvent comprises dichloromethane.

In one aspect of the invention, the suitable solvent comprises dichloromethane, and the inlet temperature of the spray dryer is between about 80° C. and about 105° C.

To form the feed solution, the drug is dissolved or dispersed in the suitable solvent at a concentration between about 0.1% w/v to about 10% w/v, and preferably between about 1% w/v and 6% w/v.

In one aspect of the present invention, crystalline Form II prepared according to the present process has a polymorphic purity of at least 90% by weight, particularly at least 95% by weight, and most particularly in excess of 98% by weight to the total 7-chloro-N,N,5-trimethyl-4-oxo-3-phenyl-3,5-dihydro-4H-pyridazino[4,5-b]indole-1-acetamide.

In a preferred aspect, the invention provides a process for preparing crystalline Form II of 7-chloro-N,N,5-trimethyl-4-oxo-3-phenyl-3,5-dihydro-4H-pyridazino[4,5-b]indole-1-acetamide as herein defined substantially free of any other polymorph of 7-chloro-N,N,5-trimethyl-4-oxo-3-phenyl-3,5-dihydro-4H-pyridazino[4,5-b]indole-1-acetamide.

In a further preferred aspect, the invention provides a process for preparing crystalline Form II of 7-chloro-N,N,5-trimethyl-4-oxo-3-phenyl-3,5-dihydro-4H-pyridazino[4,5-b]indole-1-acetamide as herein defined substantially free of impurities.

By “substantially free,” it is meant that the crystalline Form II contains less than 10% by weight, preferably less than 5% by weight, and more preferably less than 2% by weight, of any other polymorph of 7-chloro-N,N,5-trimethyl-4-oxo-3-phenyl-3,5-dihydro-4H-pyridazino[4,5-b]indole-1-acetamide or impurity or impurities.

The spray dried material according to the present invention may be combined with one or more pharmaceutically acceptable excipients generally used in the art. Such excipients may include one or more fillers; diluents, for example microcrystalline cellulose, lactose, mannitol, pregelatinized starch and the like; disintegrants, for example, sodium starch glycolate, crospovidone, croscarmellose sodium and the like; lubricants, for example, magnesium stearate, sodium stearyl fumarate and the like; sweeteners, for example, sucrose, saccharin and the like; flavoring agents, for example, peppermint, methyl salicylate, orange flavoring and the like; colorants; preservatives; buffers; solubility enhancing agents, for example, surfactants and the like; and other excipients depending on the route of administration and the dosage form used.

Accordingly, another aspect of the invention comprises the step of formulating the 7-chloro-N,N,5-trimethyl-4-oxo-3-phenyl-3,5-dihydro-4H-pyridazino[4,5-b]indole-1-acetamide crystalline Form II with one or more pharmaceutically acceptable excipients to provide a composition comprising 7-chloro-N,N,5-trimethyl-4-oxo-3-phenyl-3,5-dihydro-4H-pyridazino[4,5-b]indole-1-acetamide.

One or more pharmaceutically acceptable excipients may also be added to the solution of drug substance prior to spray drying (the “feed solution”), if the excipient(s) do not substantially decrease the relative amount of Form II prepared.

By “substantially decrease,” it is meant that the amount of crystalline Form II prepared with the pharmaceutically acceptable excipient(s) added to the feed solution is reduced by more than 25% versus the relative amount of crystalline Form II prepared without the added excipient(s). In one aspect of the invention, the pharmaceutically acceptable excipient(s) do not affect the amount of Form II prepared to an appreciable amount, as measured, for example, by Differential Scanning calorimetry and/or X-Ray Powder Diffraction as described herein.

Certain polymers may increase the stability of the amorphous form of the drug substance, and, therefore, should not be added to the feed solution in an amount which could affect the crystallinity of the spray-dried product. Accordingly, the feed solution should be substantially free of polymers, including polymers selected from the group consisting of cellulose acetate phthalate, hydroxypropyl cellulose, hydroxypropyl methyl cellulose acetate succinate, hydroxypropyl methylcellulose phthalate, polymeric polymethacrylates, and polyvinylpyrrolidone.

By “substantially free of polymers,” it is meant that the feed solution contains less than 10%, preferably less than 5%, and more preferably less than 1% of a polymer by weight relative to the drug substance. In one aspect of the invention, the feed solution contains no polymers.

In another aspect of the invention, the feed solution consists of the drug substance and a suitable solvent.

The solid state form of the spray-dried product may be confirmed by X-Ray Powder Diffraction (XPRD), Differential Scanning calorimetry (DSC), Fourier Transform Infrared Spectroscopy (FTIR), or other standard techniques known to those of skill in the art. The degree of amorphous material in the drug substance is preferably measured by Differential Scanning calorimetry and/or X-Ray Powder Diffraction; whereas X-Ray Powder Diffraction and Fourier Transform Infrared Spectroscopy are advantageously used to discriminate between Form I and Form II.

The X-ray powder diffraction pattern of Form II prepared according to the present invention is consistent with that previously observed for crystalline Form II.

The following examples will further illustrate the invention, without, however, limiting it thereto. Suitable 7-chloro-N,N,5-trimethyl-4-oxo-3-phenyl-3,5-dihydro-4H-pyridazino[4,5-b]indole-1-acetamide starting material for the herein described procedures includes, but is not limited to, 7-chloro-N,N,5-trimethyl-4-oxo-3-phenyl-3,5-dihydro-4H-pyridazino[4,5-b]indole-1-acetamide prepared by the procedures described in U.S. Pat. No. 6,395,729. Any polymorphic form of 7-chloro-N,N,5-trimethyl-4-oxo-3-phenyl-3,5-dihydro-4H-pyridazino[4,5-b]indole-1-acetamide may be used as starting material for the preparation of Form II according to the procedures described herein.

### Example 1

2.7 g of drug substance were dissolved in 150 mL of a 50/50 (by volume) mixture of dichloromethane (DCM) and ethanol (EtOH) at 20° C. The mixture was stirred for about 1 hour. The resulting clear feed solution was spray dried in a Buchi B290 Mini Spray Dryer by pumping the feed solution through a 0.7 mm nozzle (commercially available from Buchi) at approximately 2 to 3 mL/minute. The system was operated at an inlet gas temperature of 90° C. and an outlet gas temperature of approximately 60 to 70° C. The solvent was removed to isolate the Form II solid (approximately 1.8 g). XRPD showed the material to be predominantly crystalline. Both XRPD and FTIR were used to confirm the polymorphic form (FIGS. 1 and 2, labeled “90C”) as predominantly Form II.

### Example 2

1.8 g of drug substance were dissolved in 100 mL of a 50/50 (by volume) mixture of DCM and EtOH at 20° C. The mixture was stirred for about 1 hour. The resulting clear feed solution was spray dried in a Buchi B290 Mini Spray Dryer by pumping the feed solution through a 0.7 mm nozzle (commercially available from Buchi) at approximately 2 to 3 mL/minute. The system was operated at an inlet gas temperature of 100° C. and an outlet gas temperature of approximately 65 to 75° C. The solvent was removed to isolate the Form II solid (approximately 1.1 g). XRPD showed the material to be predominantly crystalline. Both XRPD and FTIR were used to confirm the polymorphic form (FIGS. 1, 2 labeled “100C”) as predominantly Form II.

### Example 3

1.8 g of drug substance were dissolved in 100 mL of a 50/50 (by volume) mixture of DCM and EtOH at 20° C. The mixture was stirred for about 1 hour. The resulting clear feed solution was spray dried in a Buchi B290 Mini Spray Dryer by pumping the feed solution through a 0.7 mm nozzle (commercially available from Buchi) at approximately 2 to 3 mL/minute. The system was operated at an inlet gas temperature of 50° C. and an outlet gas temperature of approximately 45° C.

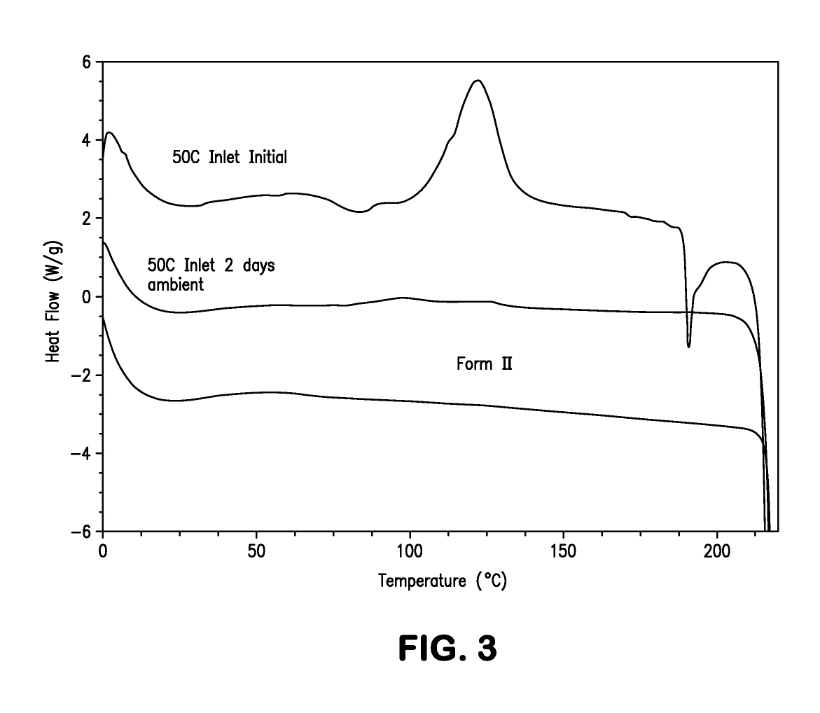

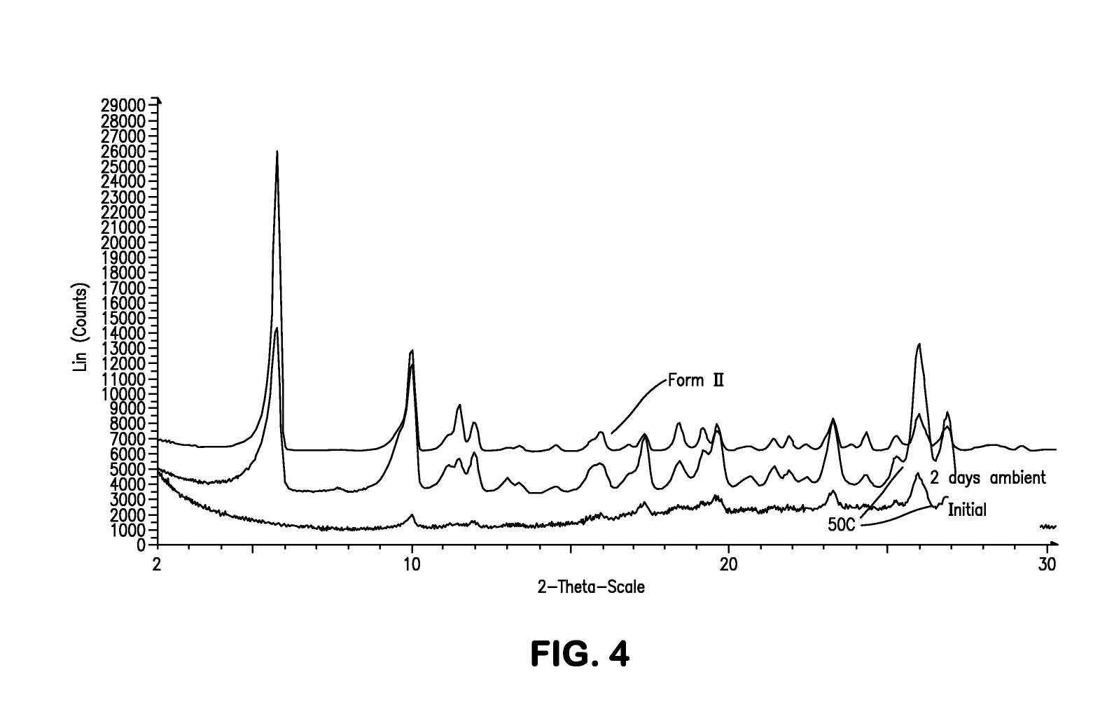

The isolated solid was analyzed by DSC (FIG. 3, labeled “50C Inlet Initial”) and XRPD (FIG. 4, labeled “50C Initial”), which indicated the presence of amorphous material. The material was aged by maintaining the sample at room temperature for two days. The solid product was reanalyzed by DSC, which indicated a predominance of crystalline Form II and a decrease in the level of amorphous material (see FIG. 3, labeled “50C Inlet 2 days ambient”). XRPD was used to confirm the polymorphic form of the aged material as predominantly Form II (FIG. 4, labeled “50C 2 days ambient”).

### Example 4

4.0 g of drug substance were dissolved in 200 mL of a 50/50 (by volume) mixture of DCM and EtOH at 20° C. The mixture was stirred for about 1 hour. The resulting clear solution was spray dried in a Buchi B290 Mini Spray Dryer by pumping the solution through a 0.7 mm nozzle (commercially available from Buchi) at approximately 2 to 3 mL/minute. The system was operated at an inlet gas temperature of 100° C. and an outlet gas temperature of approximately 70° C. The solvent was removed to isolate the solid (approximately 3.0 g). The solid was then annealed at 80° C. for 1 hour to provide the Form II solid.

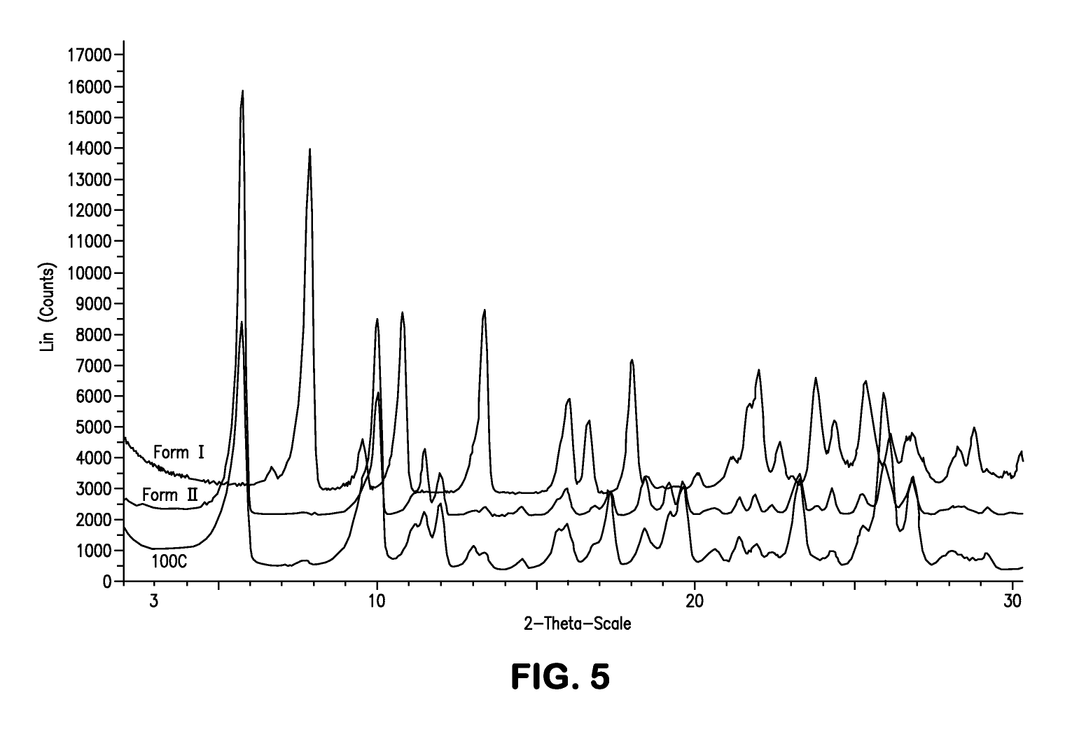

The product was analyzed by DSC, which showed no evidence of amorphous drug substance. In addition, analysis of this sample by high performance liquid chromatograph (HPLC) showed no changes in the impurity profile after the annealing process. The purity of the sample was 99.7% both before and after processing. The polymorphic form was confirmed by XRPD as predominantly Form II (FIG. 5, labeled “100C”).

### Example 5

8.7 g of drug substance was dissolved in 160 mL of DCM; the mixture was stirred for about 1 hour. The resulting clear feed solution was spray dried in a Buchi B290 Mini Spray Dryer by pumping the feed solution through a 0.7 mm nozzle (commercially available from Buchi) at approximately 2 to 3 mL/minute. The system was operated at an inlet gas temperature of 95 to 100° C. and an outlet gas temperature of approximately 70° C. The solvent was removed to isolate the solid (5.9 g). The product was annealed at 80° C. for approximately 1.5 hours.

The product was analyzed by DSC, which showed no residual amorphous solid and by XRPD, which showed the presence of substantially pure Form II and the absence of peaks indicative of Form I.

### Example 6

### Pharmaceutical Formulation

The sample prepared according the Example 5, above was used to prepare a wet granulation formulation with the following composition:

| Material | % |
|---|---|
| Product of Example 5 | 33.4 |
| Lactose | 30.0 |
| Microcrystalline cellulose (50 micron) | 30.0 |
| Sodium starch glycolate | 3.3 |
| Hydroxypropylmethylcellulose | 2.5 |
| Sodium lauryl sulfate | 0.75 |

The drug substance product of Example 5, lactose, microcrystalline cellulose (Avicel®, available from FMC BioPolymer, Philadelphia, Pa.), and sodium starch glycolate were pre-blended by screening five times with a 20 mesh screen. For the granulating fluid, the hydroxypropylmethylcellulose was dissolved in hot water at 5% concentration then cooled to 20° C. The sodium lauryl sulfate was dissolved in the cooled granulating fluid at 1.5% concentration. Approximately 50% water (relative to the weight of total solids) was used to prepare the granulation; the granulating fluid was added dropwise to the premixed powders while mixing in a mortar and pestle. The granulation was dried at 50° C. for 2 hours, and then ground in a mortar and pestle prior to analysis.

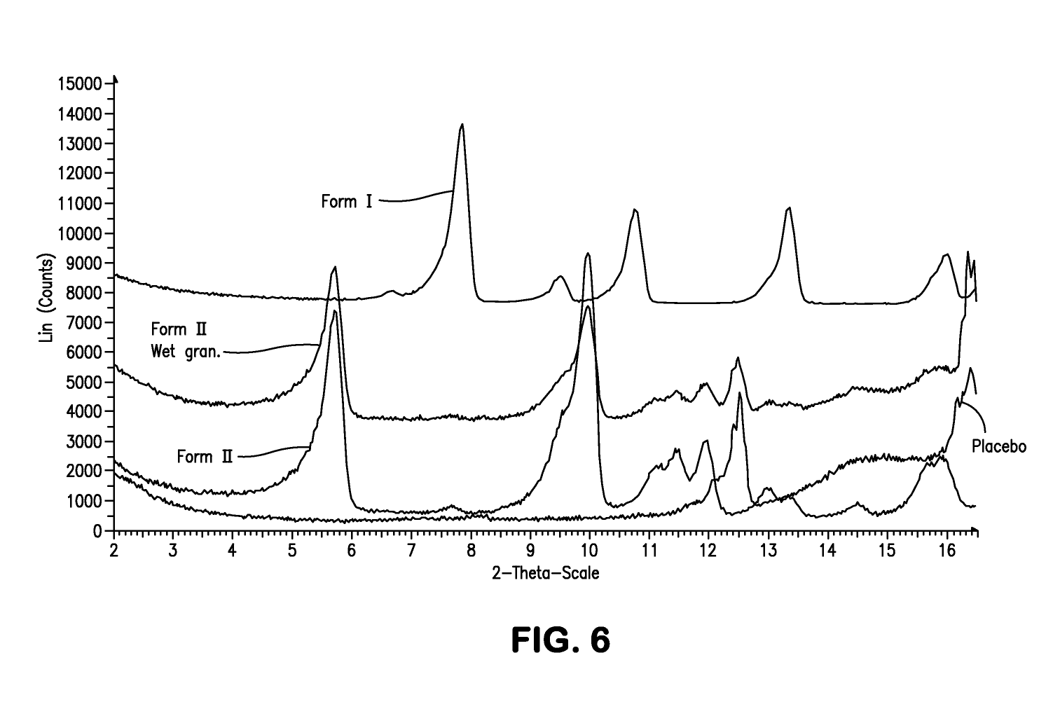

The XRPD data showed no evidence of the Form II solid converting to crystalline Form I during the wet granulation process (FIG. 6, labeled “Form II Wet Gran.”). Thus, the Form II samples produced by this process appear to be stable with respect to a pharmaceutical formulation.

The experiments below have been conducted on crystalline products prepared according to the procedures described in the Examples above, and are compared and contrasted with the solid forms of 7-chloro-N,N,5-trimethyl-4-oxo-3-phenyl-3,5-dihydro-4H-pyridazino[4,5-b]indole-1-acetamide previously described in Patent Application Publication No. WO2007/027525.

### Experimentals

### X-Ray Power Diffractometry (XRPD)

XRPD patterns were obtained with a Bruker D8® ADVANCE X-ray powder diffractometer using copper K-alpha radiation. The instrument was equipped with parallel beam optics, and the tube voltage and amperage were set to 40 kV and 40 mA, respectively. Samples were scanned at a rate of 1.0 degree/minute from 2 to 40 degrees in angle 2-theta.

A person skilled in the art will recognize that the peak locations could be slightly affected by differences in sample height. The peak locations described herein are thus subject to a variation of plus or minus (+/−) 0.15 degrees 2-theta. The relative intensities may change depending on the crystal size and morphology.

Table 1 sets forth the characteristic peak locations, d-spacings and relative intensities for the powder x-ray diffraction pattern for crystalline Form II, as provided in WO2007/027525.

TABLE 1
Characteristic XRPD Peak locations and Relative Intensities of Form II
Form II

| Angle | Spacing | Relative |
|---|---|---|
| Degrees 2θ +/− | d value | Intensity |
| 0.15° 2θ | (Angstroms) | (%) |
| 19.21 | 4.6 | 9.3 |
| 18.43 | 4.8 | 14 |
| 15.95 | 5.6 | 7.3 |
| 11.97 | 7.4 | 8.2 |
| 11.47 | 7.7 | 23.8 |
| 9.97 | 8.9 | 18.2 |
| 5.71 | 15.5 | 100 |

In particular, the peaks (expressed in degrees 2-theta +/−0.15 degrees 2-theta) at 11.47, 9.97, and 5.71 are characteristic of Form II.

Table II sets forth the characteristic peak locations, d-spacings and relative intensities for the powder x-ray diffraction pattern for crystalline Form I, as provided in WO2007/027525.

TABLE 2
XRPD Peak locations and Relative Intensities of Form I
Form I

| Angle | Spacing | Relative |
|---|---|---|
| Degrees 2θ +/− | d value | Intensity |
| 0.15° 2θ | (Angstroms) | (%) |
| 18.05 | 4.9 | 23.1 |
| 16.67 | 5.3 | 24.8 |
| 16.00 | 5.5 | 25.8 |
| 15.79 | 5.6 | 25.3 |
| 13.36 | 6.6 | 31.1 |
| 10.79 | 8.2 | 24.8 |
| 7.85 | 11.2 | 100 |

The pattern for Form II shows a unique pattern relative to Form I. A characteristic peak at 5.71 degrees (2 theta) is observed in the pattern for Form II, but is not seen in the pattern for Form I. The absence of a peak at approximately 10.79 degrees, a characteristic Form I peak, indicates that significant levels of Form I are not observed in the pattern for Form II.

FIG. 1 shows the XRPD patterns of Form II prepared according to Examples 1 and 2 compared to the XRPD pattern of Form II prepared as previously described in WO2007/027525, and to crystalline Form I.

The XRPD patterns of the solid forms prepared according to Examples 1 and 2 (labeled “90C” and “100C”, respectively) show no evidence of Form I and are consistent with Form II, as previously described.

FIG. 4 shows the XRPD pattern for the sample prepared according to Example 3, at the time of isolation and after 2 days at ambient temperature (˜20° C.). The material increased in the content of Form II and decreased in amorphous content after aging.

FIG. 5 shows the XRPD pattern for the sample prepared according to Example 4 by spray drying (labeled “100C”) and annealing. The pattern is consistent with the pattern of Form II as previously described.

FIG. 6 shows the XRPD pattern of the wet granulation prepared according to Example 6 using Form II prepared according to Example 5. There is no evidence of Form I generation in the XRPD pattern after wet granulation. The data indicates that Form II is stable with respect to wet granulation.

### Fourier Transform Infrared Spectroscopy (FTIR)

Fourier Transform IR spectra were obtained with a Nicolet 750 Magna brand system. The drug substance was ground with dry potassium bromide (KBr) at a concentration of 1 mg drug substance/100 mg KBr and compressed into a disk (200 mg) at 12,000 lbs for analysis.

FTIR spectra (see FIG. 2) showed that Form II of 7-chloro-N,N,5-trimethyl-4-oxo-3-phenyl-3,5-dihydro-4H-pyridazino[4,5-b]indole-1-acetamide has peaks at approximately 1656, 1643 to 1644, 1595, 1522, 1489, 1454, 1397, 1325, and 1282 cm⁻¹. In particular, the peaks at 1656 and 1643 to 1644 cm⁻¹ are characteristic of Form II, as previously described.

A comparison of the IR spectra (FIG. 2) of crystalline Form I and crystalline Form II showed two distinct patterns. Specifically, the amide region for Form II shows unique and characteristic peaks at approximately 1643 to 1644 cm⁻¹ and 1656 cm⁻¹, which are not present in the spectrum for Form I. In addition, the Form I band at approximately 1307 cm⁻¹ is not observed in any of the Form II samples described above.

The FTIR spectra of the solid forms prepared according to Examples 1 and 2 (labeled “90C” and “100C”, respectively) show no evidence of Form I and are consistent with Form II, as previously described.

### Differential Scanning Calorimetry (DSC)

DSC scans were performed using a TA Instruments brand differential scanning calorimeter. The system was calibrated with indium prior to use. The samples were encapsulated in aluminum pans (lids pierced). The DSC thermogram was acquired at a linear heating rate of 100° C. per minute. Amorphous drug substance can be isolated by quench cooling of a melt. The resulting material, upon reheating, exothermically recrystallizes between about 80 and about 160° C. The absence of a recrystallization peak can be used to show a high degree of crystallinity. Thus, DSC is utilized to determine the absence of amorphous material.

The DSC scan for Form II, as described in WO2007/027525, shows conversion (transformation exotherm) to a higher melting crystalline form in the region of 200° C. to 220° C., followed by a melting endotherm that is consistent with the melting point of Form I. The shape of the DSC thermogram in the temperature region where conversion occurs is likely dependent upon heating rate, particle size and sample weight. In general, DSC is not useful for detection of Form I in a sample containing predominately Form II.

The DSC scans for the solid forms prepared according to Example 3 show initial presence of amorphous material prior to aging (FIG. 3, labeled “50C Inlet Initial”). The DSC scan for the solid prepared according to Example 3, after aging (FIG. 3, labeled “50C Inlet 2 days ambient”) is consistent with crystalline Form II as previously characterized (FIG. 3, labeled “Form II”). The change to Form II was confirmed by XRPD (FIG. 4). The rate of change and the level of amorphous material would be expected to be variable at lower temperatures (e.g., 50° C.) due to the inherent instability of amorphous material.

### High Performance Liquid Chromatography (HPLC)

The HPLC method parameters utilized in Example 4 were as follows: System HP 1050, column-Waters C8 Symmetry Shield 5 um (4.6×150 mm), Mobile phase (“A”): 0.05% formic acid in water, Mobile phase (“B”): 0.05% formic acid in acetonitrile, injection volume: 10 μl, wavelength: 258 nm, and flow rate: 0.8 mL/minute. A gradient method was used according to the time table below.

| Time | % A | % B |
|---|---|---|
| 0 | 50 | 50 |
| 8 | 50 | 50 |
| 23 | 40 | 60 |
| 35 | 50 | 50 |

The purity of the sample prepared according to Example 4 was 99.7% before and after processing.

What is claimed is:

1. A process for preparing crystalline Form II of 7-chloro-N,N,5-trimethyl-4-oxo-3-phenyl-3,5-dihydro-4H-pyridazino[4,5-b]indole-1-acetamide, said process comprising spray drying a solution comprising 7-chloro-N,N,5-trimethyl-4-oxo-3-phenyl-3,5-dihydro-4H-pyridazino[4,5-b]indole-1-acetamide to form crystalline Form II, wherein said solution is substantially free of polymers.

2. The process according to claim 1 wherein the crystalline Form II exhibits an X-ray diffraction pattern comprising a peak at about 5.71 degrees 2-theta.

3. The process according to claim 2, wherein the X-ray diffraction pattern further comprises peaks at about: 11.47 and 9.97 degrees 2-theta.

4. The process according to claim 2, wherein the X-ray diffraction pattern further comprises peaks at about: 19.21, 18.43, 15.95, and 11.97 degrees 2-theta.

5. The process according to claim 1 wherein the crystalline Form II is substantially free of any other polymorph of 7-chloro-N,N,5-trimethyl-4-oxo-3-phenyl-3,5-dihydro-4H-pyridazino[4,5-b]indole-1-acetamide.

6. The process according to claim 1, wherein the spray drying comprises processing the solution through a spray dryer having an inlet temperature between about 50° C. and about 130° C.

7. The process according to claim 6, wherein the inlet temperature is between about 90° C. and about 120° C.

8. The process according to claim 1, wherein the concentration of 7-chloro-N,N,5-trimethyl-4-oxo-3-phenyl-3,5-dihydro-4H-pyridazino[4,5-b]indole-1-acetamide is between about 0.1% and about 10% (w/v).

9. The process according to claim 1, wherein the concentration of 7-chloro-N,N,5-trimethyl-4-oxo-3-phenyl-3,5-dihydro-4H-pyridazino[4,5-b]indole-1-acetamide is between about 1% and about 6% (w/v).

10. The process according to claim 1, wherein the solution further comprises one or more pharmaceutically acceptable excipients.

11. The process according to claim 1, further comprising formulating the crystalline Form II with one or more pharmaceutically acceptable excipients to provide a composition comprising 7-chloro-N,N,5-trimethyl-4-oxo-3-phenyl-3,5-dihydro-4H-pyridazino[4,5-b]indole-1-acetamide.

12. The process according to claim 1, comprising the steps of:
a) dissolving 7-chloro-N,N,5-trimethyl-4-oxo-3-phenyl-3,5-dihydro-4H-pyridazino[4,5-b]indole-1-acetamide in a suitable solvent to form a feed solution;
b) atomizing the feed solution; and
c) removing the solvent to form a solid.

13. The process according to claim 12 wherein the suitable solvent comprises one or more solvents selected from the group consisting of dichloromethane, chloroform, ethanol, methanol, 2-propanol, ethyl acetate, acetone, and dimethyl acetamide.

14. The process according to claim 12 wherein the suitable solvent comprises ethanol and dichloromethane.

15. The process according to claim 12 wherein the suitable solvent comprises dichloromethane.

16. The process according to claim 12 further comprising the step of aging the solid.

17. The process according to claim 12 wherein the 7-chloro-N,N,5-trimethyl-4-oxo-3-phenyl-3,5-dihydro-4H-pyridazino[4,5-b]indole-1-acetamide is dissolved in the suitable solvent at a temperature between about ambient temperature and the boiling point of the suitable solvent.

18. The process according to claim 12 wherein the 7-chloro-N,N,5-trimethyl-4-oxo-3-phenyl-3,5-dihydro-4H-pyridazino[4,5-b]indole-1-acetamide is dissolved in the suitable solvent at about ambient temperature.

19. A process for preparing crystalline Form II of a compound of Formula (A): 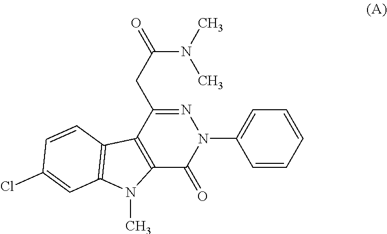
said process comprising:
a) dissolving the compound in a suitable solvent to form a feed solution, wherein the feed solution does not contain a polymer selected from the group consisting of cellulose acetate phthalate, hydroxypropyl cellulose, hydroxypropyl methyl cellulose acetate succinate, hydroxypropyl methylcellulose phthalate, polymeric polymethacrylates, and polyvinylpyrrolidone;
b) pumping the feed solution through an atomizer into a solvent removal system;
c) removing the solvent; and
d) isolating the solid.

20. The process according to claim 19, wherein the solvent removal system comprises a drying chamber.

21. The process according to claim 19, wherein the solid is isolated in a cyclone.
